(19) United States
(12) Patent Application Publication
(10) Pub. No.: US 2026/0183037 A1
(43) Pub. Date: Jul. 2, 2026

(54) CRYOABLATION CATHETER WITH VARYING CROSS-SECTIONAL SHAPE

(71) Applicant: Medtronic Ireland Manufacturing Unlimited Company, Dublin (IE)

(72) Inventor: Carlos H. Lima, Santa Rosa, CA (US)

(21) Appl. No.: 19/129,470

(22) PCT Filed: Nov. 7, 2023
(86) PCT No.: PCT/EP2023/080982

§ 371 Date: May 13, 2025

Related U.S. Application Data
(60) Provisional application No. 63/384,270, filed on Nov. 18, 2022.

(51) Int. Cl. A61B 18/02; A61B 18/00

(52) U.S. Cl. CPC A61B 18/02; A61B 2018/00172; A61B 2018/0022; A61B 2018/00577; A61B 2018/00821; A61B 2018/0212; A61B 2018/0262

(57) ABSTRACT

A cryoablation catheter includes a distal cryoshaft having a first cross-sectional shape, an inflatable balloon coupled to the distal cryoshaft, and a proximal cryoshaft having a second cross-sectional shape. The second cross-sectional shape is different than the first cross-sectional shape. The distal cryoshaft is coupled to the proximal cryoshaft at a rapid exchange joint. The cryoablation catheter further includes an inflow pipe disposed within both the proximal cryoshaft and the distal cryoshaft. The inflow pipe is coupled to the inflatable balloon.

### FIELD

This application claims the benefit of U.S. Provisional Patent Application Ser. No. 63/384,270, filed Nov. 18, 2022, the entire content of which is incorporated herein by reference.

### BACKGROUND

The present technology is generally related to cryoablation catheters.

Renal denervation and similar balloon cryotherapies are performed by inflating a balloon with a refrigerant to remove heat from surrounding tissues. The balloon is supported by a catheter which provides the refrigerant to the balloon to perform the therapy. Introduction of the refrigerant to the balloon causes the balloon to expand, and the temperature within the balloon is monitored throughout the therapy process. The catheter includes a distal portion connected to the balloon to provide the refrigerant and a proximal portion connected to the distal portion that provides the refrigerant from a refrigerant storage.

### SUMMARY

The techniques of this disclosure generally relate to the use of a proximal cryoshaft of a cryoablation catheter having a cross-sectional shape that is different than a cross-sectional shape of a distal cryoshaft of the cryoablation catheter. The cross-sectional shape of the proximal cryoshaft may maximize additional space, and may increase an exhaust area of the cryoablation catheter for improved performance during cryoablation therapy. The cross-sectional shape of the proximal cryoshaft may also increase the overall structural support of the proximal cryoshaft by providing a greater outer diameter and a greater inner diameter of the proximal cryoshaft, and allowing for a tighter balloon profile during cryoablation therapy. A smaller diameter from a baseline of the distal cryoshaft to a diameter of an inflow pipe of the distal cryoshaft and a greater diameter of the proximal cryoshaft allows for a lesser volume of the balloon to be achieved a similar profile. The pressure and temperature within the balloon decreases due to the increased exhaust area of the proximal cryoshaft that provides the lesser volume of the balloon corresponding to a tighter balloon profile.

In one aspect, the present disclosure provides a cryoablation catheter including a distal cryoshaft having a first cross-sectional shape, an inflatable balloon coupled to the distal cryoshaft, and a proximal cryoshaft having a second cross-sectional shape. The second cross-sectional shape is different than the first cross-sectional shape. The distal cryoshaft is coupled to the proximal cryoshaft at a rapid exchange joint. The cryoablation catheter further includes an inflow pipe disposed within both the proximal cryoshaft and the distal cryoshaft. The inflow pipe is coupled to the inflatable balloon.

Further disclosed herein is a cryoablation catheter that includes a distal cryoshaft having a first cross-sectional shape, an inflatable balloon coupled to the distal cryoshaft, and a proximal cryoshaft having a second cross-sectional shape, wherein the second cross-sectional shape is different than the first cross-sectional shape, wherein the distal cryoshaft is coupled to the proximal cryoshaft at a rapid exchange joint, wherein the cryoablation catheter further includes an inflow pipe disposed within both the proximal cryoshaft and the distal cryoshaft, and wherein the inflow pipe is coupled to the inflatable balloon.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a perspective view of a cryoablation catheter according to one example.

FIG. 2 is a close-up perspective view of a distal cryoshaft of the cryoablation catheter of FIG. 1, and a rapid exchange joint.

FIG. 3 is a close-up perspective view of the rapid exchange joint.

FIG. 4 is a schematic, front cross-sectional view of the cryoablation catheter, illustrating the distal cryoshaft.

FIG. 5 is a schematic, front cross-sectional view of the cryoablation catheter, illustrating the proximal cryoshaft.

FIG. 6 is a close-up perspective view of a rapid exchange joint in accordance with another example, and a proximal cryoshaft in accordance with another example, the proximal cryoshaft having a different cross-sectional shape than the cross-sectional shape of the distal cryoshaft.

FIGS. 7A-7C are schematic, front cross-sectional views of the cryoablation catheter, illustrating different cross-sectional shapes of the proximal cryoshaft according to different examples.

FIG. 8 is a schematic, front cross-sectional view of the cryoablation catheter, illustrating a comparison of cross-sectional shapes of the proximal cryoshaft.

FIG. 9 is a schematic system diagram of the cryoablation catheter.

### DETAILED DESCRIPTION

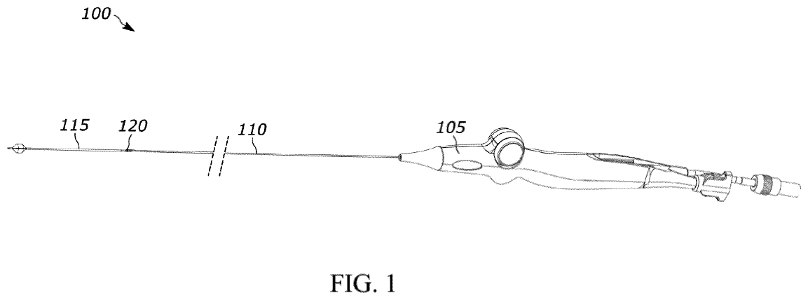

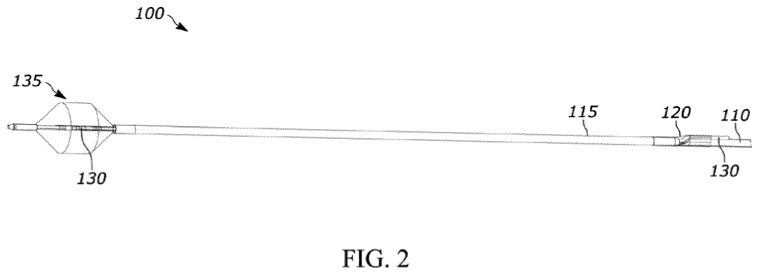

With reference to FIGS. 1-5, a medical device, illustrated as a cryoablation catheter 100, includes a handle 105, a proximal cryoshaft 110 coupled to the handle 105, and a distal cryoshaft 115 coupled to the proximal cryoshaft 110. The distal cryoshaft 115 is sized to be inserted into the vasculature of a patient. The distal cryoshaft 115 is coupled to the proximal cryoshaft 110 at a rapid exchange joint 120. The rapid exchange joint 120 is positioned to secure the proximal cryoshaft 110 to the distal cryoshaft 115 while maintaining the transition of components of the cryoablation catheter 100 between the proximal cryoshaft 110 and the distal cryoshaft 115.

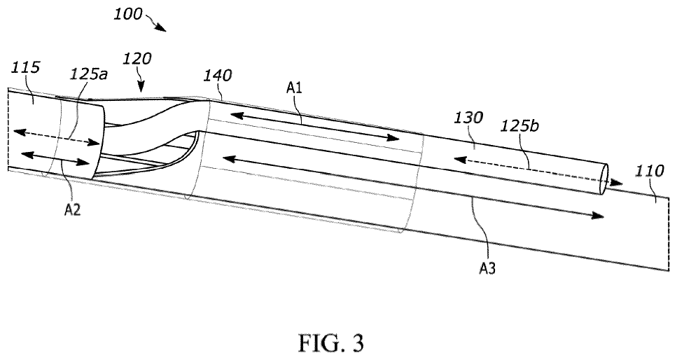

With continued reference to FIG. 1-5, in the illustrated example the cryoablation catheter 100 further includes a guidewire 125. The guidewire 125 is shaped to ensure proper alignment of the components of the cryoablation catheter 100 through the proximal cryoshaft 110 and the distal cryoshaft 115. The cryoablation catheter 100 also includes an inner member 130. A portion of the guidewire 125 extends through the inner member 130. The inner member 130 may extend to a distal end of the cryoablation catheter 100, and may be surrounded in part by a balloon 135 at a distal end of the cryoablation catheter 100. In the illustrated example, the balloon 135 is coupled to the distal cryoshaft 115, and is selectively inflatable to perform cryoablation therapy. The cryoablation catheter 100 delivers a refrigerant to the balloon 135 (e.g., through ports) to inflate and cool the balloon 135 during the cryoablation therapy. The inner member 130 may include a first portion disposed within the distal cryoshaft 115 and a second portion extending outside of the distal cryoshaft 115 (e.g., extending proximal to a portion of the distal cryoshaft 115 as illustrated in FIG. 3). While FIG. 3 illustrates an inner member 130 that extends proximally out of the rapid exchange joint 120, in some examples the inner member 130 may be trimmed or otherwise modified, such that the inner member 130 does not extend proximally out of the rapid exchange joint 120.

With reference to FIGS. 3-5, the guidewire 125 of the illustrated example includes a first portion 125a disposed within the distal cryoshaft 115 (e.g., within the portion of the inner member 130 that is within the distal cryoshaft 115) and a second portion 125b disposed outside of both the distal cryoshaft 115 and the proximal cryoshaft 110. The guidewire 125 transitions from the first portion 125a to the second portion 125b at the rapid exchange joint 120. As illustrated in FIG. 3, the first portion 125a of the guidewire 125 extends through the first portion of the inner member 130 within the distal cryoshaft 115, and the second portion 125b of the guidewire 125 extends proximally out of the second portion of the inner member 130, and alongside the proximal cryoshaft 110.

With continued reference to FIG. 3, a portion of the inner member 130 extends along a first longitudinal axis A1. The distal cryoshaft 115 extends along a second longitudinal axis A2. The second longitudinal axis A2 extends between the balloon 135 and the rapid exchange joint 120. The proximal cryoshaft 110 extends along a third longitudinal axis A3. The third longitudinal axis A3 extends between the rapid exchange joint 120 and the handle 105. In some examples, the first longitudinal axis Al is parallel to both the second longitudinal axis A2 and the third longitudinal axis A3. In some examples, the second longitudinal axis A2 is co-linear with the third longitudinal axis A3. Additionally, in some examples, the rapid exchange joint 120 further includes an elastomeric jacket 140. The elastomeric jacket 140 extends over the proximal cryoshaft 110 and the distal cryoshaft 115 to secure the connection between the proximal cryoshaft 110 and the distal cryoshaft 115 made by the rapid exchange joint 120. The elastomeric jacket 140 provides sealed protection for the rapid exchange joint 120 such that the proximal cryoshaft 110 and the distal cryoshaft 115 are not exposed to contaminants during cryoablation therapy.

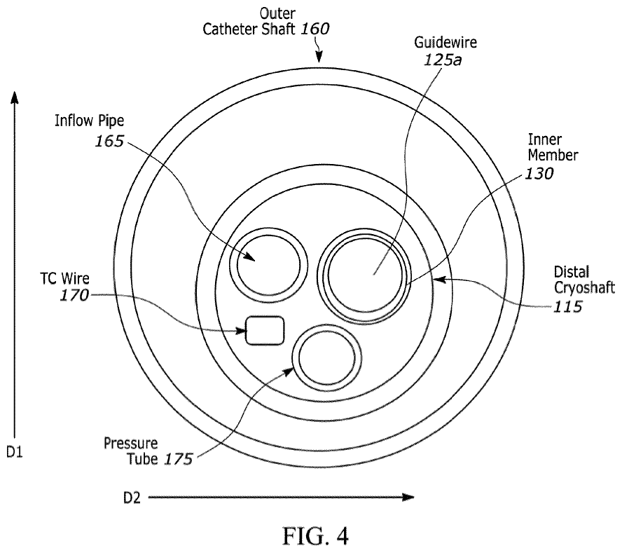

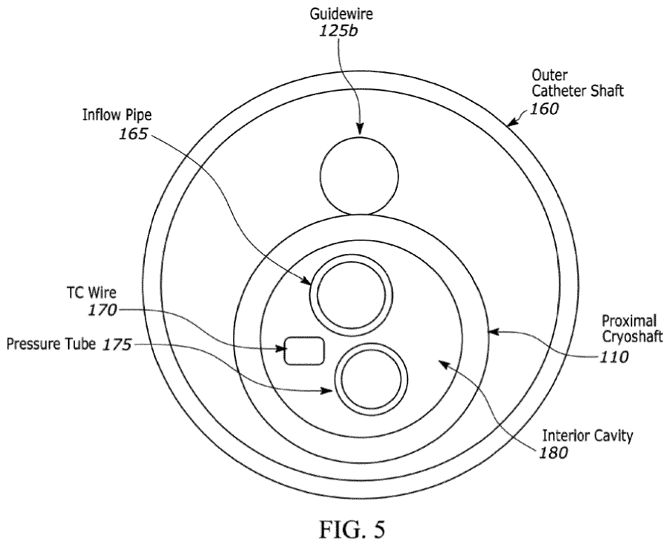

With reference to FIGS. 4 and 5, the proximal cryoshaft 110 and the distal cryoshaft 115 may each be disposed within an outer catheter shaft 160. In the illustrated example, and with reference to FIG. 4, a first cross-sectional shape of the distal cryoshaft 115 has a height measured along a first direction D1 and a width measured along a second direction D2. The second direction D2 is perpendicular to the first direction D1. In some examples, the height of the first cross-sectional shape is the same as the width of the first cross-sectional shape (e.g., the first cross-sectional shape is a circular cross-sectional shape). In other examples, the height of the first cross-sectional shape is different than the width of the first-cross-sectional shape. As illustrated in FIG. 4, the distal cryoshaft 115 may house the guidewire 125, the inner member 130, an inflow pipe 165, a thermocouple wire 170, and a pressure tube 175. In some examples, the distal cryoshaft 115 may include a greater or fewer number of components than those listed. As described above, the guidewire 125 is disposed within the inner member 130 within the distal cryoshaft 115 (e.g., the first portion 125a of the guidewire 125 extends through the inner member 130 within the distal cryoshaft 115). The inflow pipe 165 may introduce the refrigerant during cryoablation therapy, and may extend to a position within the balloon 135. In some examples, the inflow pipe 165 may include a plurality of inflow ports (not shown) that deliver the refrigerant to a volume of space within the balloon 135 during use. The inflow ports are, for example, radial openings in the inflow pipe 165 and direct the refrigerant radially outward toward the balloon 135. The refrigerant flows through the inflow pipe 165 of the distal cryoshaft 115 as a liquid and undergoes a liquid-gas phase change when passing through the inflow ports and entering the balloon 135. The phase change results in rapid expansion of the now gaseous refrigerant, thereby decreasing the temperature within the balloon 135 and inflating the balloon 135 (e.g., near a distal end of the cryoablation catheter 100). Due to the delicate nature of a cryoablation therapy, a thermocouple wire 170 (i.e., a TC wire) may be disposed within the balloon 135, and extend through the distal cryoshaft 115 to allow for monitoring of the internal temperature of the balloon 135. The pressure tube 175, including a pressure sensor, may be disposed within the balloon 135 and extend through the distal cryoshaft 115 to allow for monitoring of the internal pressure of the balloon 135. The guidewire 125 and the inner member 130 may align and secure the inflow pipe 165, the TC wire 170, and the pressure tube 175 within the distal cryoshaft 115.

With reference to FIG. 5, in some examples a portion of the proximal cryoshaft 110 has the same cross-sectional shape as the distal cryoshaft 115. As the guidewire 125 transitions from the first portion 125a to the second portion 125b, through the rapid exchange joint 120, the second portion 125b is positioned between the proximal cryoshaft 110 and the outer catheter shaft 160. Positioning the second portion 125b of the guidewire 125 between the proximal cryoshaft 110 and the outer catheter shaft 160 allows for greater space for the interior components of the proximal cryoshaft 110, and provides a hollow interior cavity 180. The proximal cryoshaft 110 may also house the inflow pipe 165, the thermocouple wire 170, and the pressure tube 175. In some examples, the proximal cryoshaft 110 may include a greater or fewer number of components than those listed. The hollow interior cavity 180 allows the proximal cryoshaft 110 to transport exhaust gas from the cryoablation therapy proximally out of the cryoablation catheter 100.

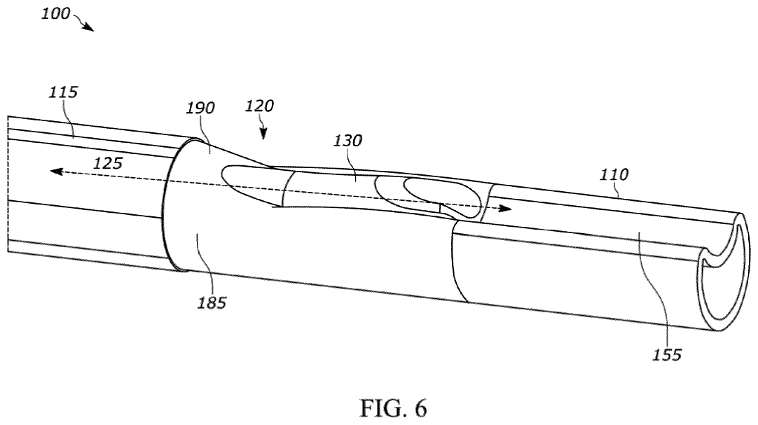

While FIGS. 1-5 illustrate a proximal cryoshaft 110 having a circular cross-sectional shape that matches the circular cross-sectional shape of the distal cryoshaft 115, the cross-sectional shape of the proximal cryoshaft 110 may be different than the cross-sectional shape of the distal cryoshaft 115. For example, and with reference to FIGS. 6-8, the proximal cryoshaft 110 may have a non-circular shape (e.g., a crescent shape, a flat top shape, an oblong and/or swaged shape, or other shape). The shape may permit a greater amount of exhaust gas to be transported out of the cryoablation catheter 100 than with the circular shape in FIGS. 1-5.

With reference to FIG. 6, in some examples the rapid exchange joint 120 includes an elongate, tubular member 185 having a ramped wall 190. The tubular member 185 may be coupled to both the proximal cryoshaft 110 and the distal cryoshaft 115, or may be formed integrally as part of one or both of the proximal cryoshaft 110 and the distal cryoshaft 115. In some examples, the ramped wall 190 extends at an oblique angle relative to the first longitudinal axis Al to connect the proximal cryoshaft 110 and the distal cryoshaft 115 via the rapid exchange joint 120.

With continued reference to FIG. 6, in the illustrated example the inner member 130 (and the guidewire 125 therein) passes through the elongate tubular member 185 and the ramped wall 190. The cross-sectional shape of the proximal cryoshaft 110 may be crescent-shaped, and/or have an outer surface defining a groove 155. The inner member 130 (and the second portion 125b of the guidewire 125 therein) may be disposed at least partially within the groove 155. In some examples, the groove 155 at least partially defines the cross-sectional shape of the proximal cryoshaft 110, such that the second portion 125b of the guidewire 125 extends parallel to the proximal cryoshaft 110 along the first longitudinal axis A1.

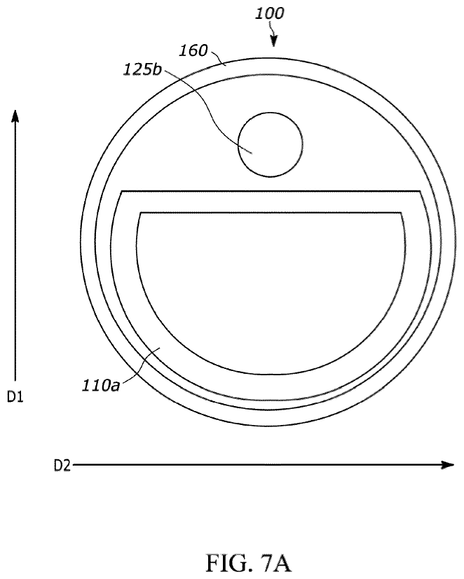

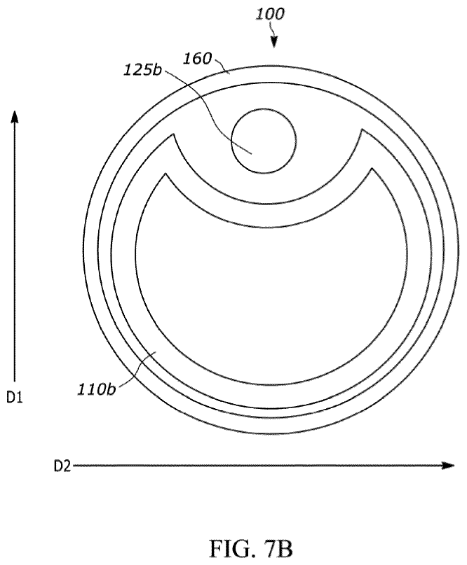

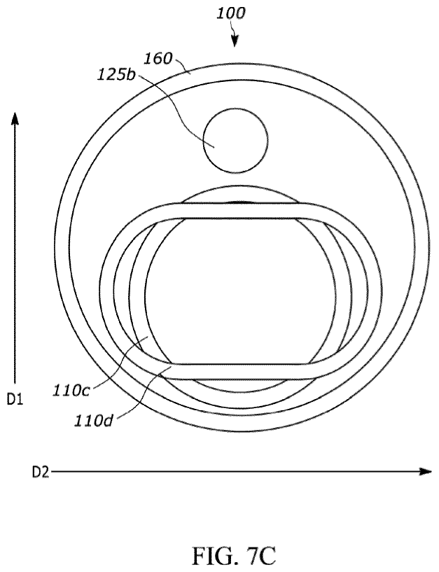

With reference to FIGS. 7A-7C, and as described above, the cross-sectional shape of the proximal cryoshaft 110 may any of a number of different cross-sectional shapes. FIGS. 7A-7C illustrate various examples of proximal cryoshafts 110a, 110b, 110c, and 110d having different shapes. Each shape has a height measured along the first direction D1 and a width measured along the second direction D2. The second direction D2 is perpendicular to the first direction D1. In some examples, the width of the second cross-sectional shape is greater than the height of the second cross-sectional shape (e.g., the second cross-sectional shape of the proximal cryoshaft 110 is a non-circular cross-sectional shape). As illustrated in FIGS. 7A-7C, the guidewire 125 is positioned between each proximal cryoshaft 110a, 110b, 110c, 110d, and the outer catheter shaft 160. In the illustrated examples, the proximal cryoshaft 110a has a flat top cross-sectional shape. In contrast, the proximal cryoshaft 110b has a crescent-shaped cross-sectional shape. In contrast, the proximal cryoshaft 110d has an oblong (e.g., swaged) cross-sectional shape. In some examples, the cross-sectional shape is that of an ellipse. In some examples, the cross-sectional shape remains constant along an entirety of the proximal cryoshaft 110. In other examples, the cross-sectional shape of the proximal cryoshaft 110 changes along the proximal cryoshaft 110. For example, in some examples, the cross-sectional shape may change from a circular shape (proximal cryoshaft 110c) to an oblong (e.g., swaged) shape (proximal cryoshaft 110d) as the proximal cryoshaft 110 extends toward the rapid exchange joint 120.

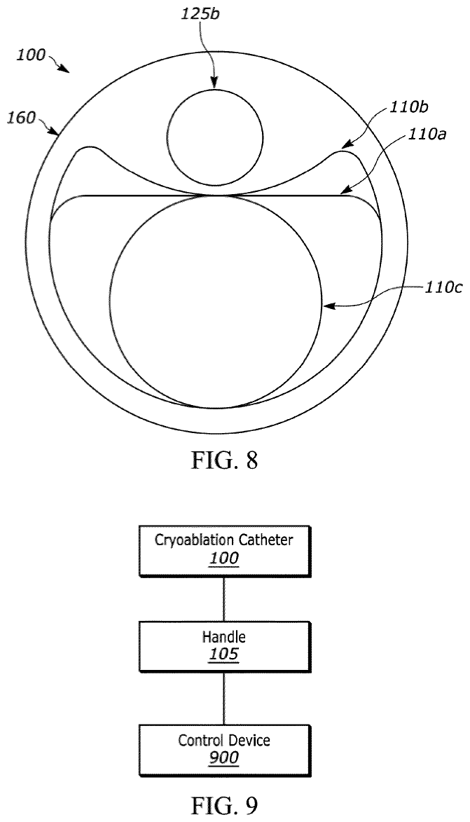

With reference to FIG. 8, these different cross-sectional shapes of the proximal cryoshaft 110 may provide an increase in exhaust area within the proximal cryoshaft 110 to transport exhaust gas, as compared to the proximal cryoshaft seen in FIGS. 1-5. The increased exhaust area may improve performance of the cryoablation catheter 100 during cryoablation therapy. For example, a greater exhaust area may provide greater vacuum power to decrease the pressure and the temperature in the balloon 135 during cryoablation therapy. As shown in FIG. 8, the guidewire 125 is positioned between the different second cross-sectional shapes of the proximal cryoshaft 110 and the outer catheter shaft 160. The flat top shape 110a and the crescent shape 110b are shown in comparison to a standard circular cross-sectional shape of the proximal cryoshaft 110. The standard circular cross-sectional shape of the proximal cryoshaft 110 in FIGS. 1-5 may have an outer diameter, for example, of 0.040″. The flat top shape of the modified proximal cryoshaft 110a may increase the overall size of the proximal cryoshaft 110, providing an increase in exhaust area within the proximal cryoshaft 210a. In some examples, the flat top shape may provide a 64% increase in exhaust area, or for example between 60% and 70%. The crescent shape 110b may provide an 80% increase in exhaust area, or for example between 70% and 90%. Although not shown, the oblong (e.g., swaged) shape may provide a 20% increase in exhaust area, or for example between 10% and 30%. Other examples include other values and ranges of values for the increase in exhaust area.

In some examples, the different (and for example larger) cross-sectional shape of the proximal cryoshaft 110 increases the overall structural support of the proximal cryoshaft 110 (e.g., by providing a greater outer diameter and a greater inner diameter of the proximal cryoshaft 110, and allowing for a tighter balloon profile during cryoablation therapy). The pressure and temperature within the balloon 135 may decrease due to the increased exhaust area of the proximal cryoshaft 110.

With reference to FIG. 9, the cryoablation catheter 100 may be used in various settings, and in combination with one or more of the handle 105 and a control device 900. In some examples, the proximal cryoshaft 110 is coupled to the handle 105. The handle 105 is coupled to the control device 900 (e.g., with wiring and/or one or more conduits for delivery of the refrigerant). The handle 105 may include one or more valves (e.g., check valves), or other features that control movement of the refrigerant flowing through the inflow pipe 165, and/or control movement of a portion or portions of the cryoablation catheter 100 itself. In some examples, no handle 105 is provided. Instead, the proximal cryoshaft 110 of the cryoablation catheter 100 is coupled (e.g., directly) to the control device 900. In some examples, the control device 900 is a large, stand-alone reusable console (e.g., with storage for the refrigerant, venting for the refrigerant, a display or monitor, and/or other features). In other examples, the control device 900 is a smaller, reusable console (e.g., without a full display or monitor, but with onboard electronics to control flow of the refrigerant, and with storage for the refrigerant or a connector for connection to a container of refrigerant). In yet other examples, the control device 900 is a disposable, handheld device for use in controlling flow of the refrigerant, and may contain for example a connector for connection to a container of refrigerant.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

Although various aspects and examples have been described in detail with reference to certain examples illustrated in the drawings, variations and modifications exist within the scope and spirit of one or more independent aspects described and illustrated.
Example 1. A cryoablation catheter comprising: a distal cryoshaft having a first cross-sectional shape; an inflatable balloon coupled to the distal cryoshaft; a proximal cyroshaft having a second cross-sectional shape, wherein the second cross-sectional shape is different than the first cross-sectional shape, and wherein the distal cryoshaft is coupled to the proximal cryoshaft at a rapid exchange joint; and an inflow pipe disposed within the proximal cryoshaft and the distal cryoshaft wherein the inflow pipe is coupled to the inflatable balloon.
Example 2. The cryoablation catheter of Example 1, wherein the first cross-sectional shape is a circular cross-sectional shape, and wherein the second cross-sectional shape is a non-circular cross-sectional shape.
Example 3. The cryoablation catheter of Example 1 or of any of Examples 1 or 2, wherein the second cross-sectional shape has a height measured along a first direction, and a width measured along a second direction that is perpendicular to the first direction, wherein the width is greater than the height.
Example 4. The cryoablation catheter of Example 1 or of any of Examples 1-3, wherein the second cross-sectional shape defines an ellipse.
Example 5. The cryoablation catheter of Example 1 or of any of Examples 1-3, wherein the second cross sectional shape is crescent-shaped.
Example 6. The cryoablation catheter of Example 1 or of any of Examples 1-5, further compromising an inner member having a first portion disposed within the distal cryoshaft, and a second portion disposed outside of the distal cryoshaft.
Example 7. The cryoablation catheter of Example 6, further comprising a guidewire having a first portion disposed within the distal cryoshaft, and a second portion disposed outside of both the distal cryoshaft and the proximal cryoshaft, wherein the guidewire transitions from the first portion to the second portion at the rapid exchange joint, wherein the first portion of the guidewire extends through the first portion of the inner member, and wherein the second portion of the guidewire extends proximally out of the second portion of the inner member.
Example 8. The cryoablation catheter of Example 7, wherein the rapid exchange joint includes an elongate tubular member coupled to both the distal cryoshaft and the proximal cryoshaft, wherein the elongate tubular member includes a ramped wall, and wherein the inner member extends through the ramped wall.
Example 9. The cryoablation catheter of Example 8, wherein the inner member extends along a longitudinal axis, and wherein the ramped wall extends at an oblique angle relative to the longitudinal axis.
Example 10. The cryoablation catheter of Example 9, wherein the longitudinal axis is a first longitudinal axis, wherein the distal cryoshaft extends along a second longitudinal axis and the proximal cryoshaft extends along a third longitudinal axis, wherein the first longitudinal axis is parallel to both the second longitudinal axis and the third longitudinal axis.
Example 11: The cryoablation catheter of Example 1 or of any of Examples 1-10, wherein the rapid exchange joint includes an elastomeric jacket extending over both the distal cryoshaft and the proximal cryoshaft.
Example 12. The cryoablation catheter of Example 1 or of any of Examples 1-11, wherein the second cross-sectional shape changes along at least a portion the proximal cryoshaft.
Example 13. The cryoablation catheter of Example 1 or of any of Examples 1-11, wherein the second cross-sectional shape remains constant along an entirety of the proximal cryoshaft.
Example 14. The cryoablation catheter of Example 1 or of any of Examples 1-6, further comprising a guidewire having a first portion disposed within the distal cryoshaft, and a second portion disposed outside of both the distal cryoshaft and the proximal cryoshaft, wherein the guidewire transitions from the first portion to the second portion at the rapid exchange joint, wherein the second portion of the guidewire extends parallel to the proximal cryoshaft.
Example 15. The cryoablation catheter of Example 14, wherein the proximal cryoshaft has an outer surface defining a groove, and wherein the second portion of the guidewire is disposed at least partially within the groove.
Example 16. The cryoablation catheter of Example 1 or of any of Examples 1-15, further comprising an outer catheter shaft, wherein the distal cryoshaft and the proximal cryoshaft are each disposed within the outer catheter shaft.
Example 17. The cryoablation catheter of Example 16, further comprising a guidewire having a first portion disposed within the distal cryoshaft, and a second portion disposed outside of both the distal cryoshaft and the proximal cryoshaft, wherein the guidewire transitions from the first portion to the second portion at the rapid exchange joint, wherein the second portion of the guidewire is positioned between the proximal cryoshaft and the outer catheter shaft.
Example 18. The cryoablation catheter of Example 1 or of any of Examples 1-17, further comprising a thermocouple wire disposed within both the proximal cryoshaft and the distal cryoshaft.
Example 19. The cryoablation catheter of Example 1 or of any of Examples 1-18, wherein a portion of the proximal cryoshaft has the same cross-sectional shape as a portion of the distal cryoshaft.
Example 20. The cryoablation catheter of Example 1 or of any of Examples 1-19, wherein the proximal cryoshaft defines a hollow interior cavity configured to transport exhaust gas proximally out of the cryoablation catheter.

1. A cryoablation catheter comprising:
a distal cryoshaft having a first cross-sectional shape;
an inflatable balloon coupled to the distal cryoshaft;
a proximal cryoshaft having a second cross-sectional shape, wherein the second cross-sectional shape is different than the first cross-sectional shape, and wherein the distal cryoshaft is coupled to the proximal cryoshaft at a rapid exchange joint; and
an inflow pipe disposed within both the proximal cryoshaft and the distal cryoshaft, wherein the inflow pipe is coupled to the inflatable balloon.

2. The cryoablation catheter of claim 1, wherein the first cross-sectional shape is a circular cross-sectional shape, and wherein the second cross-sectional shape is a non-circular cross-sectional shape.

3. The cryoablation catheter of claim 1, wherein the second cross-sectional shape has a height measured along a first direction, and a width measured along a second direction that is perpendicular to the first direction, wherein the width is greater than the height.

4. The cryoablation catheter of claim 1, wherein the second cross-sectional shape defines an ellipse.

5. The cryoablation catheter of claim 1, wherein the second cross-sectional shape is crescent-shaped.

6. The cryoablation catheter of claim 1, further comprising an inner member having a first portion disposed within the distal cryoshaft, and a second portion disposed outside of the distal cryoshaft.

7. The cryoablation catheter of claim 6, further comprising a guidewire having a first portion disposed within the distal cryoshaft, and a second portion disposed outside of both the distal cryoshaft and the proximal cryoshaft, wherein the guidewire transitions from the first portion to the second portion at the rapid exchange joint, wherein the first portion of the guidewire extends through the first portion of the inner member, and wherein the second portion of the guidewire extends proximally out of the second portion of the inner member.

8. The cryoablation catheter of claim 7, wherein the rapid exchange joint includes an elongate tubular member coupled to both the distal cryoshaft and the proximal cryoshaft, wherein the elongate tubular member includes a ramped wall, and wherein the inner member extends through the ramped wall.

9. The cryoablation catheter of claim 8, wherein the inner member extends along a longitudinal axis, and wherein the ramped wall extends at an oblique angle relative to the longitudinal axis.

10. The cryoablation catheter of claim 9, wherein the longitudinal axis is a first longitudinal axis, wherein the distal cryoshaft extends along a second longitudinal axis and the proximal cryoshaft extends along a third longitudinal axis, wherein the first longitudinal axis is parallel to both the second longitudinal axis and the third longitudinal axis.

11. The cryoablation catheter of claim 1, wherein the rapid exchange joint includes an elastomeric jacket extending over both the distal cryoshaft and the proximal cryoshaft.

12. The cryoablation catheter of claim 1, wherein the second cross-sectional shape changes along at least a portion the proximal cryoshaft.

13. The cryoablation catheter of claim 1, wherein the second cross-sectional shape remains constant along an entirety of the proximal cryoshaft.

14. The cryoablation catheter of claim 1, further comprising a guidewire having a first portion disposed within the distal cryoshaft, and a second portion disposed outside of both the distal cryoshaft and the proximal cryoshaft, wherein the guidewire transitions from the first portion to the second portion at the rapid exchange joint, wherein the second portion of the guidewire extends parallel to the proximal cryoshaft.

15. The cryoablation catheter of claim 14, wherein the proximal cryoshaft has an outer surface defining a groove, and wherein the second portion of the guidewire is disposed at least partially within the groove.

16. The cryoablation catheter of claim 1, further comprising an outer catheter shaft, wherein the distal cryoshaft and the proximal cryoshaft are each disposed within the outer catheter shaft.

17. The cryoablation catheter of claim 16, further comprising a guidewire having a first portion disposed within the distal cryoshaft, and a second portion disposed outside of both the distal cryoshaft and the proximal cryoshaft, wherein the guidewire transitions from the first portion to the second portion at the rapid exchange joint, wherein the second portion of the guidewire is positioned between the proximal cryoshaft and the outer catheter shaft.

18. The cryoablation catheter of claim 1, further comprising a thermocouple wire disposed within both the proximal cryoshaft and the distal cryoshaft.

19. The cryoablation catheter of claim 1, wherein a portion of the proximal cryoshaft has the same cross-sectional shape as a portion of the distal cryoshaft.

20. The cryoablation catheter of claim 1, wherein the proximal cryoshaft defines a hollow interior cavity configured to transport exhaust gas proximally out of the cryoablation catheter.
